(19) United States
(12) Patent Application Publication
(10) Pub. No.: US 2007/0099151 A1
(43) Pub. Date: May 3, 2007

(54) Implant having integral flexible abutment portion and method for use thereof

(76) Inventors: Daniel Ilan, Nofit (IL); Ud Bar-Peled, Moelmv Kidron (IL); Zvi Miller, Tivon (IL); Nehemia Kaplan, Ramat-Gan (IL)

Correspondence Address: NATH & ASSOCIATES, 112 South West Street, Alexandria, VA, 22314, US

(21) Appl. No.: 10/558,365

(22) PCT Filed: May 20, 2004
(86) PCT No.: PCT/IL04/00434

§ 371 Date: Dec. 29, 2006

(30) Foreign Application Priority Data
May 29, 2003 (IL) 156202

(51) Int. Cl. A61C 8/00

(52) U.S. Cl. 433/173

(57) ABSTRACT

The present invention concerns an implant, in particular a dental implant, having a below-shaped adjustable bendable portion and a cavity extending throughout the bendable portion and having an opening. The invention further concerns a method wherein the implant is placed in the bone, the adjustable bending portion is bended to the desired angle and the cavity is filled with a composition that can fix the portion at a desirable angle.

### FIELD OF THE INVENTION

The present invention relates to the field of bone implants. More particularly, the present invention relates to an implant having an integral abutment portion for facilitating connection of a prosthesis to said implant, and to a method for implanting said implant into a bone of the body.

### BACKGROUND OF THE INVENTION

A dental implant is a device that is surgically attached to a patient's jawbone in order to replace one or more missing teeth. A typical dental implant includes an implant fixture that the surgeon inserts into the jawbone, and a prosthesis, which replaces at least a portion of a missing tooth. Currently, the most prevalent type of dental implant fixture is a root-form implant. As its name suggests, the root-form implant has an elongated shape reminiscent of the bone portion of a tooth. Much like roots of natural teeth, the root-form implant penetrates the gum and anchors the prosthesis to the jawbone.

The dental implant assembly also includes an abutment, which provides an interface or transition between the implant fixture and the prosthesis. Conventional abutments typically include a substantially axi-symmetric base portion, which fits into a hole formed in the implant fixture, and a conical neck portion, which projects outward from the base portion of the abutment. Besides securing the prosthesis to the implant fixture, the abutment also compensates for misalignment between the prosthesis and adjacent teeth. Misalignment can arise, for example, when the implant fixture has an orientation with respect to the gum surface that is substantially different than the adjacent teeth.

Implant assemblies employ angled abutments, as opposed to straight abutments, to account for any misalignment. Straight and angled abutments have neck portions that project outward from their base portions in directions that are, respectively, substantially parallel or non-parallel to the symmetry axes of their corresponding base portions. Therefore, if the direction or orientation of the neck portion of the abutment is represented by a longitudinal axis that intersects the symmetry axis of the base portion (or implant fixture), the resulting orientation angle is about zero for straight abutments. In contrast, an angled abutment exhibits a non-zero orientation angle. For a discussion of straight and angled abutments, see U.S. Pat. No. 5,947,733 issued to Franz Sutter et al., which is herein incorporated by reference in its entirety for all purposes.

Though widely accepted by dental practitioners, dental implants generally, and root-form implants in particular, can be problematic. For example, the neck portions of commercially available angled abutments have fixed angular displacements with respect to their base portions, which limits their usefulness. Once a patient has been fitted with an implant fixture, the dental practitioner must order an abutment having the requisite orientation angle to ensure proper alignment of the prosthesis. However, since only discrete orientation angles are available, it is often necessary to modify the abutment to achieve the requisite angular orientation, which can be a labor intensive and costly. In some cases the necessary orientation angle may be significantly greater than what is commercially available, making it difficult to attain acceptable alignment of the prosthesis.

Dental implants having adjustable orientation angles are known, but none appear to have achieved widespread use because of design deficiencies. See, for example, U.S. Pat. No. 6,500,003 issued to Nichinonni; U.S. Pat. No. 5,890,902 issued to Sapian; U.S. Pat. No. 5,662,475 issued to Mena; U.S. Pat. No. 5,599,185 issued to Greenburg; U.S. Pat. No. 5,302,125 issued to Kownacki et al.; U.S. Pat. No. 4,793,808 issued to Kirsch; and U.S. Pat. No. 4,832,601 issued to Linden, which are herein incorporated by reference in their entirety for all purposes. Most of the disclosed implants are limited to modest orientation angles of about twenty-five degrees or less, and many do not readily permit removal of the prosthesis following installation. Some of the disclosed implants also fail to provide a smooth transition between the prosthesis and the implant fixture, which results in poor soft tissue adaptation. To ensure accurate alignment of the prosthesis with adjacent teeth, current practice provides for fabricating an abutment and prosthesis from a cast of the patients mouth following insertion of the implant fixture. Some of the disclosed designs, however, do not include a mechanism for attaching the prosthesis to the abutment prior to installation, and therefore cannot take advantage of using a laboratory cast, if desired. Additionally, root-form implant designs known in the art have had a high failure rate, especially with patients with Type IV bone, and for certain types of bone resorption.

### SUMMARY OF THE INVENTION

It is thus the object of the present invention to provide an abutment portion for an implant that has an adjustable bending portion so that the angle of the abutment in respect to the bone can by altered during the implantation proceeding, and the desired angle is fixed by inserting into a hollow cavity extending the length of the abutment a position fixating composition.

This will enable the medicinal practitioner (for example the dentist) to adjust the angle of the implant “on-line” during the implanting procedure. Precisely to the exact needs, without a necessity of the technician to prepare the implants apriori at a desired angle.

By one embodiment the implant of the present invention has an abutment portion that is integrally formed with the implant. By another embodiment the abutment is a separate component that is engagable with the implant fixture. Moreover, as it will be appreciated further, the construction of the abutment of the present invention allows for the surgeon to easily manipulate its angular orientation to any needed spatial configuration and than to fix the desired configuration by filling a hollow cavity extending throughout at least a part of the length of the implant with a position fixating composition. Additional advantages of the present invention include the universality of the design, which obviates the need for the manufacturing of numerous sizes of implants or alternatively implants each having a specific angle of bending. The implant of the present invention provides a single abutment design that can accommodate a plurality of angles. Also, the method for using the abutment and implant of the present invention requires a relatively narrow and shorter drilling channel, thus avoiding potential complications which may arise.

Another aspect of the present invention relates to the process of manufacture the abutment portion of the implant in order to get the bellow with at least two grooves extending for at least 3 mm

While the implant and abutment of the present invention are described in detail in reference to a dental implant it should appreciated that the implant of the present invention could be readily adapted for use in other bones of the body, in addition to the jawbone for various orthopedic purposes.

The present invention concerns an abutment, for mounting of a prosthesis having a distal end adapted to be connected to a prosthesis and a proximal end adapted to be connected to a bone portion of an implant, the abutment comprising a adjustable bending portion, and an inner hollow cavity that extends throughout at least a portion of the length of the abutment, the cavity having an opening at said distal end.

By one embodiment the adjustable bending portion has a bellow type construction.

By another embodiment the adjustable bending portion has a joint-like construction that may be a ball and socked joint, a knuckle joint, a rotary joint or a hinge-like joint.

Thus, the present invention further relates to an implant for being implanted into a bone of the body comprising;

(a) a bone portion for being inserted into a bone of the body;

(b) an abutment attached to said bone portion and having a distal end adapted to be connected to a prosthesis, the abutment having a adjustable bending portion;

and an inner hollow cavity that extends through at least a portion of the length of the abutment, the cavity having an opening at said distal end.

By one embodiment the adjustable bending portion has a bellow type construction.

By another embodiment the adjustable bending portion has a joint-like construction that may be a ball and socked joint, a knuckle joint a rotary joint or a hinge-like joint.

The hollow cavity may extend only throughout the length of the abutment portion or may extend also through the bone portion.

The term “abutment” in accordance with the invention refers to a part of the implant (which may be integral or separate there from) on which a prosthesis such as an artificial tooth is subsequently mounted. This part extends out of the bone.

The term “bone portion” refers to the part of the implant that is present inside the bone either in a specially drilled hole or in the hole of a previously extracted tooth (if it is to be used in dentistry).

The abutment may be integral with the bone portion (constituting together the implant fixture) or may be a separate portion attachable to the implant fixture by any manner known in the art, such as by a screw mechanism constituted of a threaded internal bore for connection through a screw. A ball-and-socket joint may be employed. Attachment of the two parts may filter be carried out by the use of laser which is used for welding of the two parts.

The abutment has a cavity that extends to a portion of its length preferably through all the length of the abutment. The cavity length should be such that once the cavity is filed with a position fixing composition, (when the bending portion is at a desired configuration), it will have sufficient strength to withhold a prosthesis. The cavity has an opening at the distal end of the abutment enabling to fill the cavity with the position fixing composition.

According to preferred embodiments of the present invention, the bone portion has an outer surface, said outer surface being non-smooth, for providing a scaffold for bone integration. In one preferred embodiment, the bone portion has a plurality of external threads located on said outer surface. In other preferred embodiments, the bone portion has a plurality of slots located on said outer surface and extending around the outer circumference thereof. It is appreciated that other designs are also possible for increasing the outer surface area of the bone portion. Further according to preferred embodiments of the present invention, the bone portion has a plurality of holes extending from the outer surface to the inner hollow cavity. These holes may have any appropriate size. As it will be described further, the holes allow for the mixing of polymer compositions located on the exterior and interior of the implant.

The bone portion may be anchored in the hole drilled in the bone, or in a hole of a previously extracted tooth, by mechanical force (inserted forcibly into the bone), or alternatively anchored with the aid of a hardening, anchoring compositions.

Any acceptable means known in the art may be employed for allowing mounting of the prosthesis onto the abutment through said distal end adapted for connection with the prosthesis. In one preferred embodiment, the distal end has a threaded internal bore for allowing connection via a screw. A ball-and-socket joint may also be employed in other embodiments. Any suitable connection may be used.

Still further according to preferred embodiments of the present invention, the adjustable bending portion of the abutment comprises an outer surface, said outer surface comprising plurality of grooves. Preferably, the grooves are rectangular shaped, though they may have other appropriate shape as well. It is appreciated that any suitable construction may be employed for enabling the adjustable bending portion to be bendable in a bellow-type or (drinking) straw-like manner. In the preferred embodiment having grooves, said grooves extend to less then half of the outer circumference of the adjustable bending portion.

The abutment and implant of the invention may be comprised of any suitable biocompatible FDA-approved dental implant material or composite of materials. For example, the abutment or implant may be formed from stainless steel. In one embodiment, the abutment or implant is comprised of titanium or nitinol. Preferably, the adjustable bending portion is comprised of flexible stainless steel or a suitable polymeric composition. In some preferred embodiments, the abutment or implant may be formed from a material having “shape memory”, such as shape memory alloys as are known in the art.

The adjustable bending portion of the abutment may have complete freedom for bending, i.e. bending at an angle of 0-90°.

Preferably, the adjustable bending portion of the abutment is adapted for being adjusted between to angles between 0-25 degrees with respect to the central vertical axis of said neck region. An angle of up to 25 degrees is preferable for the dental applications. However for other orthopedic purposes larger bending angles may be required.

By one alternative the bending is continuous to all possible angles between 0-90°, preferably between 0-25°.

By another option the bending is carried out in a step-like manner, i.e. the bellow-like shape, or the joint-like bending, has semi-flexible folds distributed at predetermined distances from each other so that the bending of each consecutive fold advances the bending of the neck to the next “step” which is a further predefined angle. For example, the folding of the bellow may be constructed so that each bending of a fold tilts the neck an additional 5°.

According to preferred embodiments of the present invention, the adjustable bending portion is comprised of flexible stainless steel.

Additionally according to preferred embodiments of the present invention, the abutment or implant further comprises at least one drug incorporated therein. The drug may be selected from, for example, anti-inflammatory agents, antibacterial agents, antimycotic agents, antibiotics, and bone-regrowth stimulants.

The abutment or implant of the present invention can be useful for surgeries performed all bone of the body, and thus can greatly improve the overall efficiency and ease of performance of such surgeries. This includes, for example, procedures performed on the jawbone, the hipbone, the spinal column, the shoulder bone, facial bone, cranial bones, and the knee.

Additionally according to preferred embodiments of the present invention, wherein the implant is used in dentistry, the implant further comprises a healing cap. Said healing caps are well known in the art and serve to promote proper tissue growth around the prosthesis and the gum-line.

Moreover according to preferred embodiments of the present invention when used in dentistry, the external diameter of the implant may be in the range of 2.0-6.0 mm is approximately 3.20 millimeters.

The implant length may be variable in accordance with the size of the hole in the bone and is not restricted to a specific length.

Further according to preferred embodiments of the present invention, the implant has a total length of approximately 15-25 millimeters. Preferably, the bone portion has a length of about 7 millimeters and the abutment portion has a length of about 9-12 millimeters.

The present invention also relates to a method for performing dental implant surgery, using a dental implant having an abutment which may be an integral part of the attached implant or a “stand alone” component to the implant. The dental implant is comprised of a bone portion and an abutment portion having an adjustable bending portion attached to the bone portion. The adjustable bending portion has an accordion-type construction (bellow-shaped). The dental implant further comprises an inner hollow cavity that extends through the bone portion and the abutment portion. Said inner hollow cavity in the abutment and in the implant extends through both the abutment and the implant is for filling with a position fixing composition. After the bendable portion of the abutment has been bent into the desired angle, the hollow cavity is filled with a composition capable of hardening that can fix the abutment at the desired angle for prolonged periods of time.

The method comprises the steps of:

(a) forming a hole in the root of the mandible or maxilla bone of a patient;

(b) affixing the bone portion of the dental implant of the invention into the hole;

(c) bending the adjustable bending portion of the abutment portion of the dental implant so as to achieve the appropriate angular configuration;

(d) filling the inner hollow cavity with a position fixing composition,;

(e) allowing the position fixing composition to harden so as to fix said appropriate angular configuration.

Optionally the method of the invention has an additional step (f):

(f) mounting a temporary or permanent dental prosthesis to the dental implant.

The mounting of the dental prosthesis may take place immediately after the implanting procedure (“immediate loading”), or at a time period up to several months after the implanting procedure so as to enable bone growth (osteo-integration) around and where applicable into the bone portion of the implant.

Further according to preferred embodiments of the present invention, the step of mounting comprises fixing (for example, via screwing or gluing) the dental prosthesis into a threaded internal bore of the abutment portion. It is appreciated that any suitable connection may be employed between the prosthesis and the abutment.

The affixing of the bone portion into the bone in step (b) above, may be achieved by one of two options:

By one option the bone portion is inserted, or screwed, by force into the hole and maintained in the hole by mechanical force. Growth of bone tissue around and possibly into the bone portion further serves to anchor the implant in the bone. It has been found that at times the bone growth which is enhanced by the mechanical pressure applied on the bone.

By another option the affixing is achieved by placing, in the space between the bone portion of the wall of the hole an anchoring composition that once hardened can anchor the implant in place.

Still further according to preferred embodiments of the present invention, the method also comprises allowing the position fixing composition to enter and fill at least part of the inner hollow cavity of the dental implant. The inner hollow cavity should extend beyond the adjustable bending portion so as to provide sufficient support to the desired configuration when the position fixing composition hardens. It may extend through the full length of the implant or through part of its length as long as it fills a region beyond the adjustable bending portion. It is appreciated that the bone portion of the implant preferably has a non-smooth surface such that, where an anchoring composition is used and placed between the bone portion and the bone (according to the second option), said composition incorporates with the implant. In some embodiments, the bone portion comprises a plurality of holes such that said anchoring composition is allowed to enter inside of the hollow cavity of the implant in order to strengthen the anchoring. It is appreciated that the use of the anchoring composition in the dental implant procedure facilitates the efficient incorporation of the implant into the bone, and the proper tissue in growth.

According to preferred embodiments of the present invention, the position fixing composition is selected from the group consisting of: polylactic acid, polyglycolic acid, polyglactin, polydioxanone, polyglyconate, and all copolymers thereof.

Additionally according to preferred embodiments of the present invention, the anchoring composition is selected from the group consisting of: polylactic acid, polyglycolic acid, polyglactin, polydioxanone, polyglyconate, and all copolymers thereof.

It is appreciated that the aforementioned method is suitable for use in any surgery performed on a bone of the body wherein the wet bleeding environment in and around a hole formed in the bone makes placing an implant of the conventional type difficult. The implant and method of the present invention thus greatly improve the ease and convenience of a plurality of different surgeries and also raise the success rate and efficiency of said surgeries

### BRIEF DESCRIPTION OF THE DRAWINGS

The present invention will now be described, by way of example only, with reference to the accompanying drawings, wherein:

FIG. 1A is a perspective side view of a dental implant having an integral abutment portion, according to certain preferred embodiments of the present invention; FIG. 1B is a perspective side view of an abutment and bone portion engagable together to form a dental implant, according to certain preferred embodiments of the present invention.

FIG. 2 is a cross sectional side view of the dental implant illustrated in FIG. 1A.

FIG. 3 shows a perspective side view of a dental implant having an integral abutment portion and having a screw-like bone portion.

### DESCRIPTION OF SPECIFIC EMBODIMENTS

It is appreciated that the detailed description provided is meant only to illustrate certain preferred embodiments of the present invention. It is in no way meant to limit the scope of the invention, as set out in the claims.

While the abutment, implant and method of the present invention will be described in detail with preferred embodiments that are specific to dental implants, it is to be appreciated that the present invention can be useful for any bone surgery, in orthopedics, bone reconstruction, the correction of birth defects, facial trauma, hip, knee, or shoulder replacement, etc.

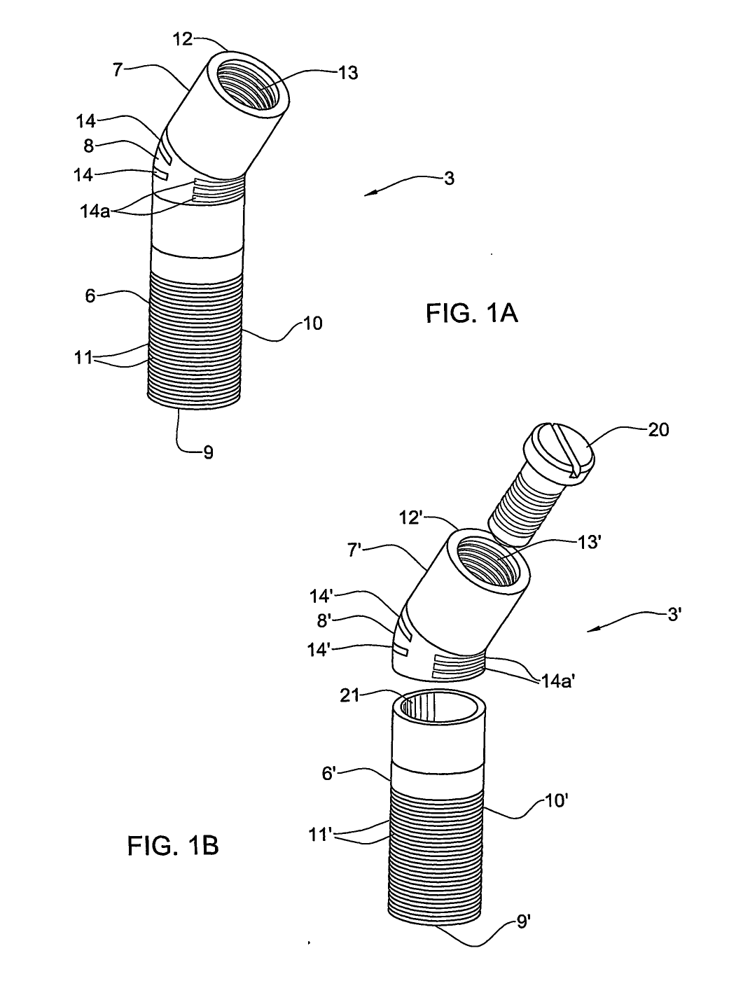

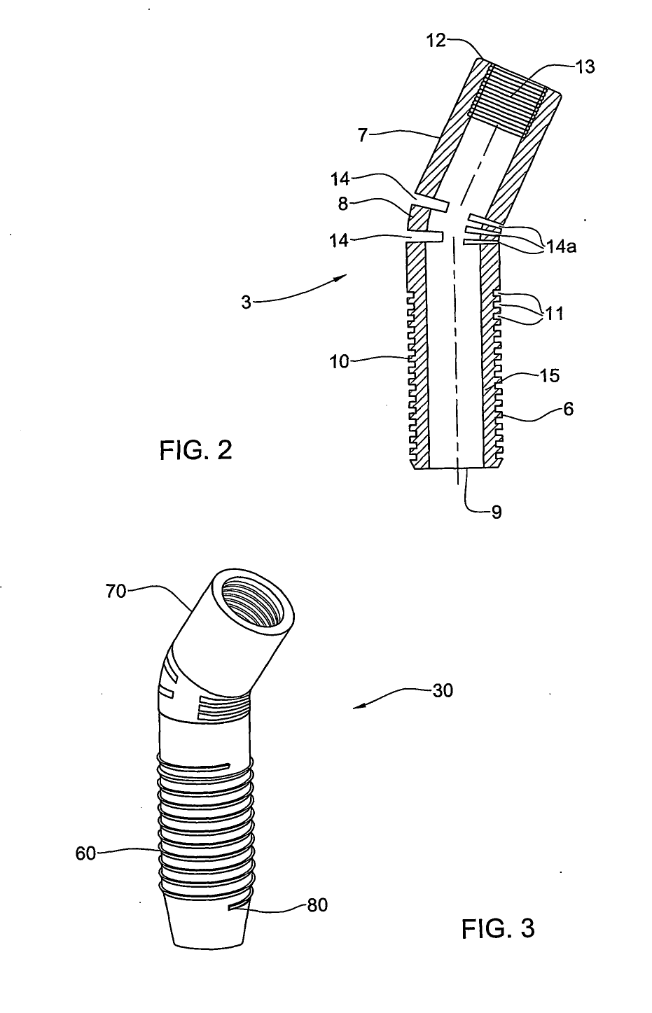

Referring to FIG. 1A, FIG. 1B in combination with FIG. 2. In FIG. 1A the dental implant (3) has abutment portion (7), which in the figure is integral with the dental implant. FIG. 1B shows a dental implant (3′) which has two parts that can be assembled together (i.e. are not a prior integral): a bone portion (6′) and abutment portion (7′). The two parts can be assembled by screw and hole fixture (20, 21), respectively. In the following description, identical elements in FIGS. 1A and 1B will be denoted by the same numbers, those of 1B with a prime (′) indication. A unique feature of the dental implant of the present invention is that the abutment portion (7) or (7′) has a adjustable bending portion (8) (8′) having bellowed flexible construction that allows the surgeon to easily set the angle of the abutment with respect to the implant for allowing for proper alignment of the dental prosthesis on top of the gum of the patient. The abutment portion is adapted for being manipulated so as to achieve angles of 0-90 degrees, preferably 0-25 degrees.

Now to describe the invention in detail, the dental implant (3) or (3′) includes a bone portion (6) or (6′) that is shaped and sized so as to fit into the root of the jawbone after a hole has been created in said bone. The outer surface (10) or (10′) of the bone portion (6) or (6′) is preferably non-smooth, so as to increase the surface area of said outer surface (10) or (10′). In the preferred embodiment shown, the outer surface (10) or (10′) of the bone portion (6) or (6′) comprises a plurality of slots (11) or (11′). The slots (11) or (11′) may be rectangular, as shown, or they may have any other suitable construction for increasing the surface are of the bone portion. The slots preferably (11) or (11′) extend around the entire circumference of the bone portion (6) or (6′). In FIG. 2 in accordance with one embodiment, the outer surface (10) or (10′) also comprises a plurality of small holes (15) or (15′) extending from the outside of the bone portion (6) or (6′) to the inner hollow cavity (9) or (9′) of the implant, for allowing fluid communication between the interior and exterior of the implant.

The dental implant (3) further comprises an abutment portion (7) as an integral part thereof, or dental implant 3′ comprises abutment portion 7′ as an assembled part. Said abutment portion (7) or (7′) includes a bellowed adjustable bending portion (8) or (8′) that is attached to the bone portion (6) or (6′) of the implant. The adjustable bending portion (8) or (8′) preferably includes a plurality of grooves (14) or (14′) for bestowing on the abutment its flexible properties. The grooves (14) or (14′) are preferably rectangular shaped, though it is appreciated that they may have other suitable configurations as well. In the preferred embodiment illustrated, the adjustable bending portion (8) or (8′) includes a first set of grooves (14) or (14′), located on one side of the abutment, and a second set of grooves (14a) or (14a′), located on the second side of the abutment, each of said sets extending less than halfway around the circumference of the adjustable bending portion (8) or (8′). There may be any number of grooves on each side, in order to provide an appropriate degree of flexibility to the abutment.

In the embodiment illustrated, the first set of grooves comprises two grooves and the second set of grooves comprises three grooves. It is appreciated that the design of the bellowed region may vary in different preferred embodiments, but any suitable design that bestows the desired degree of flexibility to the abutment is acceptable. For example, while the abutment and bellow in the present drawings are round, through they may have any polygonal shape such as hexagonal, octagonal, etc. It is appreciated that in other applications of the present invention, where the implant is used for a bone other than the jawbone, varying degrees of flexibility will be required. Because of the unique construction of the implant of the present invention, the implant can be readily adapted for such applications.

The abutment portion (7) or (7′) of the implant (3) or (3′) further comprises a connector portion (12) or (12′) for facilitating the connection between the abutment and a dental prosthesis. It is appreciated that the coupling between the abutment and the dental prosthesis may be accomplished through any suitable means known in the art. In the preferred embodiment illustrated, the connector portion (12) or (12′) has a threaded bore (13) or (13′) for allowing the prosthesis to be screwed into the implant. In other embodiments, for example, a ball-and-socket joint may be used. There are a wide variety of coupling means known in dentistry, as well as other areas of medicine benefiting from the implant design of the present invention, that could be employed.

In one embodiment for the usage of the dental implant of the present invention in dental restorative surgery, the surgeon firsts creates a hole of appropriate size into the bone of the patient. As an example only, the hole drilled may be about 3.5 millimeters in diameter. Next, the surgeon fills the hole with a predetermined amount of anchoring composition which may have in addition osteo-integrative properties. The surgeon then wets the bone portion of the implant with a predetermined activator, and inserts said bone portion (6) (6′) of the dental implant (3) (3′) into the hole drilled in the bone. Next, the surgeon manipulates the abutment portion (7) (7′) of the implant (3) (3′) so as to achieve the appropriate angle orientation in relationship to the neighboring teeth and the patient's mouth. Next, the inner hollow cavity (9) (9′) of the dental implant (3) (3′) is filled with a position fixing composition (which may be the same or different from the anchoring composition). Finally, the temporary or permanent dental prosthesis is affixed on top of the implant via connection between the connector portion (12) (12′) of the abutment portion (7) (7′) and the prosthesis.

A variety of dental compositions are well-known in the art. For example, see U.S. Pat. No. 4,097,935 to Jarcho, entitled, “Hydroxylapatite Ceramic.” Any suitable compositions may be used in the method of the present invention.

Reference is made to FIG. 3 which shows another implant of the invention (30) having an abutment portion (70) and a bone portion (60). While in FIGS. 1A and 1B the bone portion was anchored into the hole initially by the hardening of the anchoring composition placed between the walls of the extracted tooth and the non smooth surface (10) or (10′). In FIG. 3 the bone portion in (60) is in the shape of screw (80) with suitable groves that is driven to the bone by force, i.e. forcibly screwing the implant into the bone.

1. An abutment, for mounting of a prosthesis, having a distal end adapted to be connected to a prosthesis and a proximal end adapted to be connected to a bone portion of an implant, the abutment comprising an adjustable bending portion that can accommodate a plurality of angles, and an inner hollow cavity that extends throughout at least a portion of the length of the abutment, the cavity having an opening at said distal end.

2. An implant having an abutment for being implanted into a bone of the body, the implant comprising:
(a) a bone portion for being inserted into a bone of the body;
(b) an abutment attached to said bone portion and having a distal end adapted to be connected to a prosthesis, the abutment having an adjustable bending portion that can accommodate a plurality of angles; and
(c) an inner hollow cavity that extends through at least the a portion of the length of the abutment, the cavity having an opening at said distal end.

3. An implant according to claim 2, wherein the abutment is integral with the bone portion.

4. An implant according to claim 2, wherein the abutment is adapted for assembly with the bone portion.

5. An implant according to claim 4 the assembly of the abutment and the bone portion is by a screw and nut mechanism.

6. An implant according to claim 2, wherein the bone portion has an outer surface, and wherein said outer surface is non-smooth.

7. An implant according to claim 2, wherein the hollow cavity further extends through said bone portion.

8. An implant according to claim 7, wherein the bone portion has a plurality of holes extending from the exterior of the bone portion to said inner hollow cavity.

9. An implant according to claim 2, wherein the adjustable bending portion comprises an outer surface, said outer surface comprising plurality of grooves.

10. An implant according to claim 2, comprised of stainless steel.

11. An implant according to claim 2, comprised of titanium.

12. An implant according to claim 2, wherein the adjustable bending portion is adapted for being adjusted to angles between 0-90 degrees with respect to the central vertical axis of said adjustable bending portion.

13. An implant according to claim 2, wherein the adjustable bending portion is adapted for being adjusted to angles between 0-25 degrees with respect to the central vertical axis of said adjustable bending portion.

14. An implant according to claim 2, wherein the adjustable bending portion is comprised of flexible stainless steel.

15. An implant according to claim 2, further comprising at least one drug incorporated therein.

16. An implant according to claim 15, wherein the drug is selected from the group consisting of: anti-inflammatory agents, antibacterial agents, antimycotic agents, antibiotics, and bone-re growth stimulants.

17. The implant of claim 2 being a dental implant.

18. A dental implant according to claim 17, further comprising a healing cap.

19. A dental implant according to claim 17, having an external diameter of about 2.0-6.0 millimeters.

20. A dental implant according to claim 17, having a length of about 15-25 millimeters.

21. A method for performing dental implant surgery, using a dental implant of claim 17, the method comprising the steps of;
a. forming a hole in the root of the mandible or maxilla bone of a patient;
b. affixing the bone portion of the dental implant as defined in claim 17 into the hole;
c. bending the adjustable bending portion of the abutment portion of the dental implant so as to achieve the appropriate angular configuration;
d. filling the inner hollow cavity with a position fixing composition;
e. allowing the position fixing composition to harden so as to fix said appropriate angular configuration.

22. A method according to claim 21, further comprising step (f): mounting a temporary or dental prosthesis to the dental implant.

23. A method according to claim 21, wherein the affixing of step (b) is achieved by force driving the bone portion into the mandible or maxilla bone of the patient.

24. A method according to claim 21, wherein the affixing of step (b) is achieved by at least partially filling the hole in the root by an anchoring composition and providing conditions allowing the anchoring composition to harden.

25. A method according to claim 22, wherein the step of mounting comprises affixing the dental prosthesis into a threaded internal bore at the distal end of the abutment portion.

26. A method according to claim 24, further comprising allowing the anchoring composition to enter and fill at least part of the inner hollow cavity of the dental implant.

27. A method according to claim 24, wherein the anchoring composition is selected from the group consisting of: polylactic acid, polyglycolic acid, polyglactin, polydioxanone, polyglyconate, and all copolymers thereof.

28. A method according to claim 21, wherein the position fixing composition is selected from the group consisting of: polylactic acid, polyglycolic acid, polyglactin, polydioxanone, polyglyconate, and all copolymers thereof.
